(19) United States
(12) Patent Application Publication
(10) Pub. No.: US 2012/0027855 A1
(43) Pub. Date: Feb. 2, 2012

(54) PHARMACEUTICAL COMPOSITIONS FOR GASTROINTESTINAL DRUG DELIVERY

(75) Inventors: Ashish Ashokrao DESHMUKH, Pune (IN); Pravin Meghrajji Bhutada, Pune (IN); Sajeev Chandran, Pune (IN); Thommandru Vijaya Kumar, Pune (IN); Shirishkumar Kulkarni, Pune (IN); Ninad Deshpanday, Pune (IN)

(73) Assignee: Lupin Limited, Mumbai (IN)

(21) Appl. No.: 13/195,526

(22) Filed: Aug. 1, 2011

Related U.S. Application Data
(63) Continuation-in-part of application No. 12/144,894, filed on Jun. 24, 2008.

(30) Foreign Application Priority Data
Jul. 6, 2007 (IN) 969/KOL/2007

(51) Int. Cl. A61K 31/65; A61P 31/04; A61K 9/20

(52) U.S. Cl. 424/465; 514/152

(57) ABSTRACT

The present invention relates to controlled release pharmaceutical formulations of active principle(s) like tetracycline-class antibiotics for providing increased residence time in the gastrointestinal tract and the process of preparing them.

This application is a Continuation-in-Part application of U.S. Ser. No. 12/144,894, filed 24 Jun. 2008, which claims benefit of Serial No. 969/KOL/2007, filed 6 Jul. 2007 in India and which applications are incorporated herein by reference. A claim of priority to all, to the extent appropriate is made.

### FIELD OF THE INVENTION

The present invention relates to controlled release pharmaceutical formulations of active principle(s) like tetracycline-class antibiotics for providing increased residence time in the gastrointestinal tract and the process of preparing them.

### BACKGROUND OF THE INVENTION

A number of drugs act in the gastrointestinal tract. Oral drug administration is by far the most preferable route for taking medications. However, on oral administration, normal or pathological stomach voiding and intestinal peristaltic movements limit the time for which a drug-releasing dosage form remains in the gastrointestinal tract or at the required site of action. Specifically, during pathological conditions such as diarrhea, peristaltic movement of the GI Tract is increased. Therefore, GI transit time of dosage forms is lesser than normal. Hence conventional dosage forms have shorter residence time at the site of absorption or at required site of action and need to be dosed frequently in order to be therapeutically effective. A rational approach to solve this problem and to enhance bioavailability and improve pharmacokinetic and pharmacodynamic profiles is to retain the drug reservoir above its absorption area or the site of action, and to release the drug in a controlled manner, for a prolonged period of time. However, controlling the GI residence time is a challenge. We have surprisingly found that it is possible to extend the GI residence time of non-systemic locally acting drugs when administered in controlled release mucoadhesive dosage forms. Drugs like (i) carbamazepine (an antiepileptic), furosemide (a diuretic), metoprolol (a beta blocker) and acyclovir (an antiviral), minocycline (antibiotic) benefit from prolonged presence at or near the locus of absorption in terms of their bioavailability characteristics including drugs that act specifically on the gastrointestinal tract (e.g. 5-aminosalicylic acid) or which are absorbed most efficiently within the intestine or colon (e.g. peptides or proteins such as insulin, interferon, calcitonin, endorphins, human growth hormone, and various hormone growth factors).

Acne affects large patient populations, and is a common inflammatory skin disorder which usually localizes in sebaceous areas of the body including on the face, back and chest. Fortunately, the disease usually disappears, and in the interval of months or years between onset and resolution, therapy, although not curative, can satisfactorily suppress the disease in the majority of patients.

Oral tetracycline-class antibiotics are frequently used in the treatment of acne. Tetracycline-class antibiotics are known to have some side effects. These side effects include vestibular symptoms such as vertigo, dizziness or blurred vision. These effects are sometimes disabling. See, Gould & Brookler, Arch. Otolarang. Vol. 96, p. 291 (1972); Williams et al., Lancet, Sep. 28, 1974, p. 144-45; Fanning & Gump, Arch. Intern. Med., Vol. 136, pp. 761-62 (1976). Headache and general malaise, along with gastro-intestinal symptoms such as the diarrhea, nausea, gas, or cramps may also occur. Dry nose and dry mouth are also occasionally encountered.

One of the oral tetracycline-class antibiotics used in the treatment of acne is minocycline hydrochloride. Oral dosage forms of minocycline hydrochloride are available commercially under various trade names. These commercial products are immediate-release oral dosage forms of minocycline hydrochloride. The dosing schedule used most frequently for treating acne using currently available immediate-release oral dosage forms is 100 mg of minocycline (free base equivalent) administered twice daily, see Leyden, J. Cutis 2006; 78 (suppl 4):4-5. However, some patients experience adverse effects with currently available immediate-release oral dosage forms, leading to reduced rates of patient compliance. See Stewart, M. et al., Cutis 2006; 78 (suppl 4):11-20.

U.S. Pat. No. 5,908,838, discloses slowly dissolving dosage forms of oral tetracycline-class antibiotics, including minocycline hydrochloride, that reduce the incidence or severity of vestibular side effects resulting from the treatment of acne.

Known bioadhesive solid dosage forms are described, for example, in GB-2,042,888 (Teijin). A slow release pharmaceutical preparation to be used adhering to the mucosa of the oral cavity (buccal) or nasal cavity comprising an active ingredient, 50 to 95% of a cellulose ether and 50 to 5% of a high molecular weight cross-linked polyacrylic acid (carboxyvinyl polymer, carbomer, carbopol).

U.S. Pat. No. 6,303,147 (Janssen) describes a bioadhesive pharmaceutical composition comprising a pharmaceutically effective amount of an active ingredient, from 80% to 98.8% (w/w) pre-gelatinized starch, and from 1% to 10% (w/w) of a hydrophilic matrix forming polymer, characterized in that the composition further comprises from 0.2% to 5% (w/w) alkaliC16-22 alkyl fumarate as a lubricant.

U.S. Pat. No. 6,306,789 (Reckitt Benckiser Healthcare) describes bioadhesive granules of carbomer and in particular to such granules containing pharmaceutical active agents suitable for sustained release into the gastrointestinal tract or for targeted delivery to the gastrointestinal mucosa.

U.S. Pat. No. 5,900,247 (Adir et compagnie) describes the bioadhesive films or patches characterized by the use: of a polymer (A) composed of one or a number of vinyl acetate/polyvinylpyrrolidone copolymers.

U.S. Pat. No. 5,472,704 (Recordati S. A.) describe composition characterized by plurality of small-size units capable of ensuring a gradual release of the active ingredient they contain the units being coated with a bioadhesive polymer layer. The composition makes it possible to keep the release controlling function separate from the function providing bioadhesion.

WO 2006/031420 (Spherics) describes bioadhesive formulation includes a multilayered core enveloped by a bioadhesive coating.

Although the development of slowly dissolving forms of minocycline hydrochloride was a significant advance in the art, there remains a long-felt need for treatments that are effective in suppressing acne but associated with fewer adverse effects than those associated with the various immediate-release oral dosage forms of minocycline hydrochloride.

### OBJECTS OF THE INVENTION

The main object of the invention is to provide controlled release pharmaceutical compositions of tetracycline-class antibiotic.

An object of the invention is to provide a novel pharmaceutical composition, which comprises a therapeutically effective amount of active principle(s) or a pharmaceutically acceptable salt or enantiomer or polymorph thereof, optionally one or more controlled release agent(s) and pharmaceutical acceptable excipient(s) thereof, wherein the composition is formulated to increase the residence time of the pharmaceutical composition and/or active principle(s) in the gastrointestinal tract.

Another object of the invention is to provide a controlled release pharmaceutical composition, which comprises a therapeutically effective amount of tetracycline-class antibiotic, one or more bioadhesive polymer(s) and one or more pharmaceutically acceptable excipient(s) thereof, wherein the composition is formulated to increase the residence time of said pharmaceutical composition and/or tetracycline-class antibiotic in the gastrointestinal tract, more particularly, the present invention relates to controlled release compositions comprising minocycline.

Another object of the invention is to provide a controlled release pharmaceutical composition of tetracycline-class antibiotic comprising: at least two entities selected from
a. controlled release entity
b. bioadhesive entity
c. optionally one or more immediate release entities;
   and one or more pharmaceutically acceptable excipient(s), wherein said composition is formulated to increase the residence time of said pharmaceutical composition and/or tetracycline-class antibiotic in the gastrointestinal tract.

Another object of the invention is to provide a novel pharmaceutical composition comprising: at least two entities wherein one entity is an immediate release or fast release and the other is controlled release wherein the composition is formulated to increase the residence time of active principle(s) in the gastrointestinal tract.

Another object of the invention is to provide a controlled release pharmaceutical composition of tetracycline-class antibiotic comprising: at least two entities wherein one entity is an immediate release and the other is controlled release wherein the composition is formulated to increase the residence time of tetracycline-class antibiotic in the gastrointestinal tract.

Another object of the invention is to provide a novel pharmaceutical composition comprising: at least two entities wherein one entity is an immediate release or fast release and the other is bioadhesive wherein the composition is formulated to increase the residence time of active principle(s) in the gastrointestinal tract.

Another object of the invention is to provide a controlled release pharmaceutical composition comprising: at least two entities wherein one entity is an immediate release or fast release and the other is bioadhesive wherein the composition is formulated to increase the residence time of tetracycline-class antibiotic in the gastrointestinal tract.

Another object of the invention is to provide a novel pharmaceutical composition comprising: at least two entities wherein one entity is controlled release and the other is bioadhesive wherein the composition is formulated to increase the residence time of active principle(s) in the gastrointestinal tract.

Another object of the invention is to provide a controlled release pharmaceutical composition comprising: at least two entities wherein one entity is controlled release and the other is bioadhesive wherein the composition is formulated to increase the residence time of tetracycline-class antibiotic in the gastrointestinal tract.

Another object of the invention is to provide a novel pharmaceutical composition in the form of a multilayer tablet comprising, a) at least one layer which comprises, a therapeutically effective amount of active principle(s) or a pharmaceutically acceptable salt or enantiomer or polymorph thereof, pharmaceutically acceptable excipient(s); wherein the said layer provides a immediate or fast release of active principle(s); and b) at least another layer which provides increased residence time of the dosage form in the gastrointestinal tract.

Another object of the invention is to provide a controlled release pharmaceutical composition in the form of a multilayer tablet comprising, a) at least one layer which comprises, a therapeutically effective amount of tetracycline-class antibiotic, one or more pharmaceutically acceptable excipient(s); wherein the said layer provides a immediate release of tetracycline-class antibiotic; and b) at least another layer which provides increased residence time of the dosage form in the gastrointestinal tract.

Another object of the invention is to provide a novel pharmaceutical composition in the form of a multilayer tablet comprising, a) at least one layer which comprises, a therapeutically effective amount of active principle(s) or a pharmaceutically acceptable salt or enantiomer or polymorph thereof, pharmaceutically acceptable excipient(s); wherein the said layer provides a controlled release of active principle(s); and b) at least another layer which provides increased residence time of the dosage form in the gastrointestinal tract.

Another object of the invention is to provide a controlled release pharmaceutical composition in the form of a multilayer tablet comprising, a) at least one layer which comprises, a therapeutically effective amount of tetracycline-class antibiotic, one or more pharmaceutically acceptable excipient(s); wherein the said layer provides a controlled release of tetracycline-class antibiotic; and b) at least another layer which provides increased residence time of the dosage form in the gastrointestinal tract.

Another object of the invention is to provide a novel pharmaceutical composition in the form of a multilayer tablet comprising, a) at least one layer which comprises, a therapeutically effective amount of active principle(s) or a pharmaceutically acceptable salt or enantiomer or polymorph thereof, pharmaceutically acceptable excipient(s); and b) at least one layer comprising another or same active principle(s) wherein layer (b) provides increased residence time of the dosage form in the gastrointestinal tract.

Another object of the invention is to provide a controlled release pharmaceutical composition in the form of a multilayer tablet comprising, a) at least one layer which comprises, a therapeutically effective amount of tetracycline-class antibiotic, one or more pharmaceutically acceptable excipient(s); and b) at least one layer comprising tetracycline-class antibiotic wherein layer (b) provides increased residence time of the dosage form in the gastrointestinal tract.

Another object of the invention is to provide a novel pharmaceutical composition in the form of a multilayer tablet comprising, a) at least one layer which comprises, a therapeutically effective amount of active principle(s) or a pharmaceutically acceptable salt or enantiomer or polymorph thereof, pharmaceutically acceptable excipient(s); wherein the said layer provides a immediate or fast release of active principle(s); and b) at least one layer comprising another or same active principle(s) wherein layer (b) provides residence time of the dosage form in the gastrointestinal tract.

Another object of the invention is to provide a controlled release pharmaceutical composition in the form of a multilayer tablet comprising, a) at least one layer which comprises, a therapeutically effective amount of tetracycline-class antibiotic, one or more pharmaceutically acceptable excipient(s); wherein the said layer provides a immediate release of tetracycline-class antibiotic; and b) at least one layer comprising tetracycline-class antibiotic wherein layer (b) provides residence time of the dosage form in the gastrointestinal tract.

Another object of the invention is to provide a novel pharmaceutical composition in the form of a multilayer tablet comprising, a) at least one layer which comprises, a therapeutically effective amount of active principle(s) or a pharmaceutically acceptable salt or enantiomer or polymorph thereof, pharmaceutically acceptable excipient(s); wherein the said layer provides controlled release of active principle(s); and b) at least one layer comprising another or same active principle(s) wherein layer (b) provides increased residence time of the dosage form in the gastrointestinal tract.

Another object of the invention is to provide a controlled release pharmaceutical composition in the form of a multilayer tablet comprising, a) at least one layer which comprises, a therapeutically effective amount of tetracycline-class antibiotic, one or more pharmaceutically acceptable excipient(s); wherein the said layer provides controlled release of tetracycline-class antibiotic; and b) at least one layer comprising another or same active principle(s) wherein layer (b) provides increased residence time of the dosage form in the gastrointestinal tract.

Another object of the invention is to provide a novel pharmaceutical composition in the form of a multilayer tablet comprising, a) at least one layer which comprises, a therapeutically effective amount of active principle(s) or a pharmaceutically acceptable salt or enantiomer or polymorph thereof, pharmaceutically acceptable excipient(s); wherein the said layer provides a immediate or fast release of active principle(s); and b) at least one layer comprising another or same active principle(s) wherein layer (b) provides controlled and/or increased residence time of the dosage form in the gastrointestinal tract.

Another object of the invention is to provide a controlled release pharmaceutical composition in the form of a multilayer tablet comprising, a) at least one layer which comprises, a therapeutically effective amount of tetracycline-class antibiotic, one or more pharmaceutically acceptable excipient(s); wherein the said layer provides a immediate release of tetracycline-class antibiotic; and b) at least one layer comprising tetracycline-class antibiotic wherein layer (b) provides controlled and/or increased residence time of the dosage form in the gastrointestinal tract.

Yet another object of the invention is to provide a novel pharmaceutical composition comprising a therapeutically effective amount of active principle(s) or a pharmaceutically acceptable salt or enantiomer or polymorph thereof, optionally one or more release controlling agent and pharmaceutical acceptable excipient(s) thereof, wherein the composition is formulated to increase the residence time of the said pharmaceutical composition and/or active principle(s) in the gastrointestinal tract, having an adhesive strength, measured as a force of detachment, of at least 100 mN when measured using advanced force gauge equipment (manufactured by Mecmesin, West Sussex, England).

Another object of the invention is to provide a controlled release pharmaceutical composition comprising a therapeutically effective amount of tetracycline-class antibiotic, one or more bioadhesive polymer(s) and one or more pharmaceutically acceptable excipient(s) thereof, wherein the composition is formulated to increase the residence time of the said pharmaceutical composition and/or tetracycline-class antibiotics in the gastrointestinal tract, having an adhesive strength, measured as a force of detachment, of at least 100 mN when measured using advanced force gauge equipment (manufactured by Mecmesin, West Sussex, England).

Another object of invention is to provide a controlled release pharmaceutical composition of minocycline, wherein composition releases from about 10% to about 35% of minocycline in one hour, from about 40% to about 85% of minocycline in six hours, measured using USP Type II dissolution apparatus in 900 ml of 0.1 N HCl at 75 rpm.

Another object of invention is to provide the method for reducing the incidence or severity of vestibular side effects resulting from the treatment of acne by the use of controlled release pharmaceutical composition of Minocycline, one or more bioadhesive polymer(s) and one or more pharmaceutically acceptable excipient(s) wherein said composition is formulated to increase the residence time of said pharmaceutical composition and/or minocycline in the gastrointestinal tract, such that:
i) said composition provides therapeutic blood concentration of minocycline over a 24 hours period
ii) said composition provides a peak blood plasma level (Cₘₐₓ) of minocycline in more than 4 hours (Tₘₐₓ).

Another object of invention is to provide the method for reducing the incidence or severity of vestibular side effects resulting from the treatment of acne by the use of controlled release pharmaceutical composition of Minocycline, one or more bioadhesive polymer(s) and one or more pharmaceutically acceptable excipient(s) wherein said composition is formulated to increase the residence time of said pharmaceutical composition and/or minocycline in the gastrointestinal tract, such that:
i) said composition provides therapeutic blood concentration of minocycline over a 24 hours period
ii) said composition provides a peak blood plasma level (Cₘₐₓ) of minocycline in about 4 to about 12 hours (Tₘₐₓ).

### BRIEF DESCRIPTION OF THE DRAWINGS

FIG. 1 is a graph illustrating the force in mN required to separate a tablet from a biological substrate.

FIG. 2 is a graph of illustrating dissolution profiles (cumulative percent drug release vs. time) for Examples 21 and 22.

### DETAILED DESCRIPTION OF THE INVENTION

The present invention relates to controlled release pharmaceutical compositions of cyclines. Particularly, present invention relates to controlled release pharmaceutical compositions of tetracycline-class antibiotic and a process of preparing them.

The present invention relates to a novel pharmaceutical composition, which comprises a therapeutically effective amount of active principle(s) or a pharmaceutically acceptable salt or enantiomer or polymorph thereof, optionally one or more controlled release agent(s) and pharmaceutical acceptable excipient(s) thereof, wherein the composition is formulated to increase the residence time of the said pharmaceutical composition and/or active principle(s) in the gastrointestinal tract.

The present invention relates to a controlled release pharmaceutical composition, which comprises a therapeutically effective amount of tetracycline-class antibiotic, one or more bioadhesive polymer(s) and one or more pharmaceutically acceptable excipient(s) thereof, wherein the composition is formulated to increase the residence time of said pharmaceutical composition and/or tetracycline-class antibiotic in the gastrointestinal tract, more particularly, the present invention relates to controlled release compositions comprising minocycline.

The pharmaceutical composition according to the invention can remain attached for desired period of time to the epithelial surface or to the mucosal membrane of the gastrointestinal tract. Since many drug compounds are absorbed exclusively in the small intestine or in a limited segment of the GI tract, it would therefore be beneficial to develop dosage forms such as sustained release dosage forms, which remains in the stomach and/or in the proximal and/or in the distal portion of the intestine for an extended period of time. The compositions of the present invention are preferably administered as once-a day. It can be administered twice a day or once a week.

Preferred group of drugs that could benefit from retained and controlled or immediate release in the gastrointestinal tract are those meant for the treatment of pathologies located in the stomach, the duodenum or the small intestine or colon.

The terms “active principle,” “drug” “active agent” “active” and “pharmacologically active agent” are used interchangeably herein to refer to a chemical material or compound which, when administered to an organism (human or animal, generally human) induces a desired pharmacologic effect. In the context of the present invention the active principles are selected from the group comprising anti-infectives, penicillins, cephalosporins, cyclines, beta-lactamase inhibitors, aminosides, quinolones, nitroimidazoles, sulfamides, antihistaminics, antiallergics, anesthetics, steroidal or non-steroidal anti-inflammatories, analgesics with local or systemic effect, antispasmodics, anticancers, diuretics, beta-blockers, antihypertensives, antianginals, antiarrhythmics, vasodilators, bradycardic agents, calcium inhibitors, sedatives, cardiotonics, antifungals, antiulceratives, vasotonics, vasoprotectants, anti-ischemics, antiemetics, anticoagulants, antithrombotics, immunosuppressants, immunomodulators, antivirals, antiretrovirals, antidiabetics, hypolipidemics, agents for combating obesity, anticonvulsants, hypnotics, antiparkinsonians, antimigraines, neuroleptics, anxiolytics, antidepressants, psychostimulants, agents for promoting memory, bronchodilators, antitussives, agents for combating osteoporosis, peptides, hormones, steroids, enzymes, enzyme inhibitors, proteins, melatonergic agonists or antagonists, hormonal agents, acidity reducing agents (e.g., buffering agents such as potassium phosphate dibasic, calcium carbonate, sodium bicarbonate, sodium and potassium hydroxide, etc.) or combinations thereof. Cyclines refers to tetracycline-class antibiotic selected from minocycline, Sumycin, Terramycin, Tetracyn, Panmycin or pharmaceutically acceptable salts thereof.

Minocycline may be in the form of a free base, an acid salt (e.g., hydrochloride salt), enantiomer or polymorph or a mixture thereof. Reference herein to “minocycline” will be understood as encompassing all such forms like salts, unless the context clearly indicates otherwise. Dosages of minocycline salts will be understood to be on the basis of the amount of minocycline free base provided thereby, and thus may be expressed as aminocycline free base equivalent dosage or amount. Minocycline salts are pharmaceutically acceptable in some embodiments, preferably minocycline hydrochloride. The term “pharmaceutically acceptable”, as used herein, refers to a drug, salt, carrier, etc., that can be introduced safely into human or an animal body (e.g., taken orally and digested, etc.).

The preferred active agents that can be used in conjunction with the present invention include rifaximin, vancomycin, mesalamine, cholestesamine, balasalazide, sulfasalazine etc.

“Therapeutically effective amount” means that the amount of active agent, which halts or reduces the progress of the condition being treated or which otherwise completely or partly cures or acts palliatively on the condition. A person skilled in the art can easily determine such an amount by routine experimentation and with an undue burden.

“Therapeutically effective amount” means that the amount of tetracycline-class antibiotic, which halts or reduces the progress of the condition like acne and symptoms associated with acne like acne vulgaris being treated or which otherwise completely or partly cures or acts palliatively on the condition. A person skilled in the art can easily determine such an amount by routine experimentation and with an undue burden.

“Controlled release,” means drug delivery system releasing the drug at a predetermined rate, locally or systemically, for a specified period of time. The term “controlled release” used in pharmaceutical compositions of invention meant release of the active ingredient from pharmaceutical composition is modified to occur at a slower rate than that from an immediate release composition. Controlled release can be used interchangeably with prolonged release, programmed release, timed release, extended release, sustained release and other such dosage forms.

“Optional” or “optionally” means that the subsequently described circumstance may or may not occur, so that the description includes instances where the circumstance occurs and instances where it does not.

By “pharmaceutically acceptable” is meant a carrier comprised of a material that is not biologically or otherwise undesirable.

“Entities” or “Entity” can be interchangeably used with granules, pellets, beads, minitablets and the like.

In an embodiment prolonged gastrointestinal residence can be obtained by using oral mucoadhesive formulation and/or by reducing gastrointestinal motility or a combination of one or more techniques.

In another embodiment, the gastroretentivity of the dosage form composition might also be achieved by delaying the gastric emptying time such as by administration of food.

The term “mucoadhesive” can be used interchangeably with “bioadhesive” and is defined as a natural or synthetic component, including macromolecules, polymers, and oligomers, or mixtures thereof, that can adhere to a subject's mucous membrane.

“Bioadhesion” or “mucoadhesion” is defined as the ability of a material to adhere to a biological tissue for an extended period of time. Bioadhesion is one solution to the problem of inadequate residence time resulting from stomach emptying and intestinal peristalsis, and from displacement by ciliary movement. For sufficient bioadhesion to occur, an intimate contact must exist between the bioadhesive and the receptor tissue, the bioadhesive must penetrate into the crevice of the tissue surface and/or mucus, and mechanical, electrostatic, or chemical bonds must form. Bioadhesive properties of polymers are affected by both the nature of the polymer and by the nature of the surrounding media.

“Residence time” is the time required for a pharmaceutical dosage form to transit through the stomach to the rectum i.e. the pharmaceutical dosage forms of the invention may have an increased retention time in the stomach and/or small and/or large intestine, or in the area of the gastrointestinal tract that absorbs the drug contained in the pharmaceutical dosage form. For example, pharmaceutical dosage forms of the invention can be retained in the small intestine (or one or two portions thereof, selected from the duodenum, the jejunum and the ileum). These pharmaceutical dosage forms as a whole, may include a bioadhesive polymeric coating that is applied to at least one surface of the dosage form.

In another embodiment, a controlled release pharmaceutical composition comprising Minocycline, one or more bioadhesive polymer(s) and one or more pharmaceutically acceptable excipient(s) wherein said composition is formulated to increase the residence time of said pharmaceutical composition and/ Minocycline in the gastrointestinal tract.

In another embodiment, a controlled release pharmaceutical composition comprising tetracycline-class antibiotic, at least two bioadhesive polymer(s) and one or more pharmaceutically acceptable excipient(s) wherein said composition is formulated to increase the residence time of said pharmaceutical composition and/or tetracycline-class antibiotic in the gastrointestinal tract.

In another embodiment, a controlled release pharmaceutical composition comprising Minocycline, at least two bioadhesive polymer(s) and one or more pharmaceutically acceptable excipient(s) wherein said composition is formulated to increase the residence time of said pharmaceutical composition and/or Minocycline in the gastrointestinal tract.

Examples of mucoadhesives or bioadhesive polymers for use in the embodiments disclosed herein include, but are not limited to, natural, semisynthetic and synthetic polymers.

Natural polymers include proteins (e.g., hydrophilic proteins), such as pectine, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic; hyaluronic acid, polyhyaluronic acid, alginic acid, sodium alginate.

When the bioadhesive polymer is a synthetic polymer, the synthetic polymer is typically selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers or mixtures thereof.

Other polymers suitable for use in the invention include, but are not limited to, methyl cellulose, ethyl cellulose, hydroxypropyl cellulose, hydroxypropylmethyl cellulose, hydroxybutylmethyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), polyvinyl acetate), polyvinyl chloride, polystyrene, polyvinyl pyrrolidone, and polyvinylphenol. Polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Another group of polymers suitable for use as bioadhesive polymers are polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymers are polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphonic acids, positively charged groups such as (protonated) amines and neutral, polar groups such as amides and imines.

Preferably, the hydrophilic groups are selected to not interfere and instead to enhance the bioadhesiveness of the polymers. In embodiments of the present invention, a pharmaceutical composition comprises an active agent and at least one swellable polymer.

Swellable polymers include, but are not limited to, a crosslinked poly(acrylic acid), a poly(alkylene oxide), a polyvinyl alcohol), a polyvinyl pyrrolidone); a polyurethane hydrogel, a maleic anhydride polymer, such as a maleic anhydride copolymer, a cellulose polymer, a polysaccharide, starch, and starch based polymers.

Polymers can be modified by increasing the number of carboxylic groups accessible during biodegradation, or on the polymer surface. The polymers can also be modified by binding amino groups to the polymer. The polymers can be modified using any of a number of different coupling chemistries available in the art to covalently attach ligand molecules with bioadhesive properties to the surface-exposed molecules of the polymeric microspheres.

Lectins can be covalently attached to polymers to render them target specific to the mucin and mucosal cell layer. The attachment of any positively charged ligand, such as polyethyleneimine or polylysine, to a polymer may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most polymers with the appropriate chemistry, such as with carbodiimidazole (CDI), and be expected to influence the binding to the gut. For example, polyclonal antibodies raised against components of mucin or else intact mucin, when covalently coupled to a polymer, would provide for increased bioadhesion. Similarly, antibodies directed against specific cell surface receptors exposed on the lumenal surface of the intestinal tract would increase the residence time when coupled to polymers using the appropriate chemistry. The ligand affinity need not be based only on electrostatic charge, but other useful physical parameters such as solubility in mucin or specific affinity to carbohydrate groups.

The covalent attachment of any of the natural components of mucin in either pure or partially purified form to the polymers generally increases the solubility of the polymer in the mucin layer. The list of useful ligands include but are not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, such as polyaspartic acid and polyglutamic acid, may also increase bioadhesiveness. The polyamino chains would increase bioadhesion by means of chain entanglement in mucin strands as well as by increased carboxylic charge.

In another embodiment, a controlled release pharmaceutical composition of tetracycline-class antibiotic comprising tetracycline antibiotic, a bioadhesive polymer and one or more pharmaceutically acceptable excipient(s) wherein said composition is formulated to increase the residence time of said pharmaceutical composition and/or tetracycline-class antibiotic in the gastrointestinal tract such that bioadhesive polymer is from about 5% to about 50% and more preferably about 5% to about 30% based on total weight of said composition.

In another embodiment, a bioadhesive controlled release pharmaceutical dosage form of the invention can have one or more coatings such as enteric coating, controlled release coating, film coating, sugar coating, bioadhesive coating. In one example, the additional coating prevents the bioadhesive dosage form from contacting the mouth or esophagus. In another example, the coating remains intact until reaching the small intestine (e.g., an enteric coating). Examples of coatings include methylmethacrylates, zein, cellulose acetate, cellulose phthalate, HMPC, sugars, enteric polymers, gelatin and shellac. Premature exposure of a bioadhesive layer or dissolution of a pharmaceutical dosage form in the mouth can be prevented with a layer or coating of hydrophilic polymers such as HPMC or gelatin.

Coating agents which are useful in the coating process, include, but are not limited to, polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, opadry, polyvinyl alcohol, copolymers of vinylpyrrolidone and vinyl acetate (e.g. marketed under the brand name of Plasdone) and polymers based on methacrylic acid such as those marketed under the brand name of Eudragit. These may be applied from aqueous or non-aqueous systems or combinations of aqueous and non-aqueous systems as appropriate.

The bioadhesive polymers discussed above can be mixed with one or more plasticizers or thermoplastic polymers. Such agents typically increase the strength and/or reduce the brittleness of polymeric coatings. The plasticizers include dibutyl sebacate, polyethylene glycol, triethyl citrate, dibutyl adipate, dibutyl fumarate, diethyl phthalate, ethylene oxide-propylene oxide block copolymers and di(sec-butyl) fumarate, thermoplastic polymers include polyesters, poly(caprolactone), polylactide, poly(lactide-co-glycolide), methyl methacrylate, cellulose and derivatives thereof such as ethyl cellulose, cellulose acetate and hydroxypropyl methyl cellulose and large molecular weight polyanhydrides.

Antitacking agents such as talc, stearic acid, magnesium stearate and colloidal silicon dioxide and the like.

Surfactants such as polysorbates and sodium lauryl sulphate and opacifying agents such as titanium dioxide and the like. All these excipients can be used at levels well known to the persons skilled in the art.

A pharmaceutical dosage form can have one or more coatings in addition to the bioadhesive polymeric coating, e.g., covering the surface of the bioadhesive coating. These coatings and their thickness can, for example, be used to control where in the gastrointestinal tract the bioadhesive coating becomes exposed.

Pharmaceutical dosage forms of the invention can be coated by a wide variety of methods. Suitable methods include compression coating, coating in a fluidized bed or a pan and hot melt (extrusion) coating. Such methods are well known to those skilled in the art.

Also suitable are rupturable coating systems, e.g., Pulsincap, that use osmotic forces of swelling from hydrophilic polymers to rupture enteric membranes to reveal underlying bioadhesive dosage form.

Alternately, non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc.

Also, coatings of polymers that are susceptible to enzymatic cleavage by colonic bacteria are another means of ensuring release to distal ileum and ascending colon. Materials such as calcium pectinate can be applied as coatings to tablets and multiparticulates and disintegrate in the lower gastrointestinal tract, due to bacterial action. Calcium pectinate capsules for encapsulation of bioadhesive multiparticulates are also available.

The pharmaceutically acceptable excipients are selected from the group comprising binders, diluents, lubricants, disintegrants, pH stabilizing agents, surfactants and glidants.

The amounts of excipient(s) employed will depend upon how much active agent is to be used. One excipient(s) can perform more than one function. Binder is one or more selected from the group comprising carbohydrates like celluloses their derivatives; starches; gums; polyvinylpyrrolidone, povidone, syrup, polyethylene oxide, polyacryl amide, poly-N-vinyl amide, sodium carboxymethyl cellulose, polyethylene glycol, gelatin, polyethylene oxide, poly propylene glycol, tragacanth, alginic acid, combinations thereof.

Diluent is one or more selected from the group comprising carbohydrates, derivatives of carbohydrates, polyols, sugar alcohols, carbonate, and sulphate or phosphate salts of inorganic metals or mixtures thereof.

Fillers or diluents, as used in the invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Disintegrants comprises but not limited to starches; clays; celluloses; alginates; gums; cross-linked polymers, e.g., cross-linked polyvinyl pyrrolidone or crospovidone, e.g., POLYPLASDONE XL, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the invention facilitates in the release of drug in the latter stage and thereby completely releasing the drug from the dosage form.

Lubricants is one or more selected from the group comprising Magnesium, Aluminium, Zinc or Calcium stearate, polyethylene glycol, mineral oil, sodium stearyl fumarate, stearic acid, hydrogenated vegetable oil, glyceryl behenate, glyceryl palmitostearate, glyceryl stearate, cornstarch, talc, calcium silicate, magnesium silicate, colloidal silicon dioxide, silicon hydrogel, and mixtures thereof.

Surfactant can be selected from ionic or non-ionic or zwitterionic surfactants. The preferred agent is copolymers composed of a central hydrophobic chain of polyoxypropylene (poly(propylene oxide)) and polyoxyethylene (poly(ethylene oxide)) that is well known as poloxamer. However, other agents may also be employed such as dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

Glidant is one or more selected from the group comprising silicon dioxide, colloidal silica, powdered cellulose, talc, tribasic calcium phosphate and mixtures thereof.

Lubricants as used in the invention comprises but not limited to Mg, Al,Ca or Zn stearate, polyethylene glycol, glyceryl behenate, mineral oil, sodium stearyl fumarate, stearic acid, hydrogenated vegetable oil and talc.

The novel pharmaceutical compositions of the present invention can further have solubilizing agents. Solubilizing agents include but are not limited to surfactants, cyclodextrin and its derivatives, lipophilic substances or any combination thereof.

Unlimiting examples of surfactants include water-soluble or water dispersible nonionic, semi-polar nonionic, anionic, cationic, amphoteric, or zwitterionic surface-active agents; or any combination thereof.

Other solubilizing agents include vitamin E and its derivatives; monohydric alcohol esters such as trialkyl citrates, lactones and lower alcohol fatty acid esters; nitrogen-containing solvents; phospholipids; glycerol acetates such as acetin, diacetin and triacetin; glycerol fatty acid esters such as mono-, di- and triglycerides and acetylated mono- and diglycerides; propylene glycol esters; ethylene glycol esters; and combinations thereof.

In another embodiment of the novel pharmaceutical composition of the present invention can further have stabilizing agents. Stabilizing agents include but are not limited to catalysts, antioxidants, adsorbents, absorbents, buffers, chelating and sequestering agents, carbonate salt of said amino acid is present as either the group I or II alkali or alkali earth metal salt and combinations thereof. A pharmaceutical composition of the invention comprise but is not limited to powders, pellets, beads, granules, tablets, compacts, sustained release formulations, capsules, microcapsules, tablets in capsules, tablets in tablets, microspheres, shear form particles, floss, and flakes or mixtures thereof. Tablets include single layered tablets, multilayered tablets, mini tablets, bioadhesive tablets, caplets, matrix tablets, tablet within a tablet, mucoadhesive tablets. Sustained release is formulation include but are not limited to matrix type controlled release, membrane diffusion controlled release, site targeted, osmotically controlled release, pH dependent delayed release, timed release, pulsatile release, hydrodynamic balanced system; powders, pellets, beads, granules for suspension.

Multi-layer tablets comprises a first, a second or/and a third layer, where each layer includes one or more excipients and optionally one or more drug like tetracycline-class antibiotic(s).

At least one layer of the tablet includes a hydrophobic excipient or hydrophilic excipient or combinations thereof.

Exemplary hydrophobic excipients include but not limited to celluloses, particularly cellulose acetate and ethyl cellulose, stearic acid, magnesium stearate, glycerol monostearate, fatty acids and salts thereof, monoglycerides, diglycerides, triglycerides, oil, colloidal silicon dioxide and talc.

Preferably, hydrophobic excipient (s) comprises Ammonio methacrylate copolymers type A and B as described in USP, methacrylic acid copolymer type A, B and C as described in USP, Polyacrylate dispersion 30% as described in Ph. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl actylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

The hydrophilic excipient (s) according to invention comprises but not limited to cellulose derivatives, alginic acid derivatives, polysaccharides, alkylene oxides or mixtures thereof.

Preferably, hydrophilic excipient (s) comprises celluloses or their salts or derivatives thereof, hydroxyethylcellulose, hydroxypropyl cellulose, hydroxypropyl methylcellulose (hypromellose), sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol, lactose.

Such tablets include one excipient present in an amount sufficient to be at least partially rate-controlling with respect to release of the drug like tetracycline-class antibiotic from the tablet.

For tablets containing two or more drugs, the drugs can both be present in one or more layers or the different drugs like tetracycline-class antibiotics are present in separate layers.

For drugs requiring absorption in the stomach and upper small intestine and/or topical delivery to these sites, particularly drugs with narrow absorption windows, bioadhesive, gastroretentive drug delivery systems are the option of choice. Drugs requiring absorption or topical delivery only in the small intestine, enteric-coated, bioadhesive drug delivery systems are preferred. For drugs requiring absorption or topical delivery only in the lower small intestine and colon enteric-coated, bioadhesive drug delivery systems are preferred.

Multi-layer or gradient tablets can be assembled in several different ways.

In one embodiment, the tablet comprises at least one controlled release layer and one bioadhesive layer, where in controlled release layer comprises one or more pharmaceutical polymers and/or pharmaceutical excipients, one or more drugs.

In another embodiment, the tablet comprises at least one controlled release layer and one bioadhesive layer, where in controlled release layer comprises one or more bioadhesive polymers and/or pharmaceutical excipients, tetracycline-class antibiotic.

In one embodiment, the tablet comprises at least one controlled release layer and one bioadhesive layer, each comprising one or more pharmaceutical polymers and/or pharmaceutical excipients, optionally one or more drugs. Such tablets can also be used to commence release of different drugs at different times, by inclusion of different drugs in separate layers.

In one embodiment, the tablet comprises at least one controlled release layer and one bioadhesive layer, each comprising one or more bioadhesive polymers and/or pharmaceutical excipients, tetracycline-class antibiotic. Such tablets can also be used to commence release of different tetracycline-class antibiotics at different times, by inclusion of different drugs in separate layers.

In one embodiment, the tablet comprises at least one solid inner layer and two solid outer layers, each comprising one or more pharmaceutical polymers and/or pharmaceutical excipients. The inner layer comprises one or more active ingredient and rate-controlling polymer. The two solid outer layers are bioadhesive.

In another embodiment, the tablet comprises at least one solid inner layer and two solid outer layers, each comprising one or more bioadhesive polymers and/or pharmaceutical excipients. The inner layer comprises one or more tetracycline-class antibiotics and rate-controlling polymer. The two solid outer layers are bioadhesive.

In one embodiment, the tablet comprises at least one solid inner layer and two solid outer layers, each comprising one or more drugs and one or more pharmaceutical polymers and/or pharmaceutical excipients. Such tablets can also be used to commence release of different drugs at different times, by inclusion of different drugs in separate layers.

In another embodiment, the tablet comprises at least one solid inner layer and two solid outer layers, each comprising one or more tetracycline-class antibiotics and one or more bioadhesive polymers and/or pharmaceutical excipients. Such tablets can also be used to commence release of different tetracycline-class antibiotics at different times, by inclusion of different tetracycline-class antibiotics in separate layers.

In another embodiment, the multi-layer tablet consists of a solid inner layer and two solid outer layers, each comprising a drug and one or more pharmaceutical polymers or pharmaceutical excipients, wherein at least one polymer or excipient is hydrophobic.

In another embodiment, the multi-layer tablet consists of a solid inner layer and two solid outer layers, each comprising tetracycline-class antibiotic and one or more bioadhesive polymers or pharmaceutical excipients, wherein at least one bioadhesive polymer or excipient is hydrophobic.

In an embodiment, the composition of the present invention comprises at least two fractions wherein one fraction is an immediate release or fast release fraction providing an immediate release of the active agent and the other fraction is an extended release fraction that releases the active agent over extended periods of time.

In another embodiment, the composition of the present invention comprises at least two fractions wherein one fraction is an immediate release fraction providing an immediate release of tetracycline-class antibiotic and the other fraction is an extended release fraction that releases tetracycline-class antibiotic over extended periods of time wherein ratio of immediate release fraction to extended release fraction is in the range of 1:9 to 2:3 and more preferably 1:3.

One or more layers of the tablet can contain permeation enhancers to provide permeability enhancement of drugs like tetracycline-class antibiotic through mucosal lining of the gastrointestinal tract (GIT). An absorption enhancer facilitates the uptake of a drug like tetracycline-class antibiotic across the gastrointestinal epithelium. Absorption enhancers include compounds that improve the ability of a drug like tetracycline-class antibiotic to be solubilized in the aqueous environment in which it is originally released and/or in the lipophilic environment of the mucous layer lining of the intestinal walls.

In still another embodiment, the multi-layer tablet is enteric coated. Optionally Eudragit FS 30D or other suitable polymer may be incorporated in coating composition to retard the release of the drug like tetracycline-class antibiotic to ensure drug like tetracycline-class antibiotic release in the colon.

It is also an object of the invention to provide the controlled release pharmaceutical composition, wherein the said composition is formulated by compressing or compacting powder, granules, pellets, beads, shear form particles, floss, or the like, or combinations thereof. The said composition of the present invention may be filled into capsule or made into a capsule, wherein the said capsule is in the form of a hard gelatin capsule or soft gelatin capsule.

It is also an object of the present invention to provide a controlled release pharmaceutical composition wherein the composition is in the form of a compressed or compacted multiparticulate composition comprising a blend of one or more types of particles, granules, pellets, beads, compacts, minitablets, shear form particles, floss, or the like, or combinations thereof, having different release characteristics or a multiparticulate composition made into a capsule or filled into a capsule.

It is also an object of the present invention to provide a pharmaceutical composition of active principle(s) like tetracycline-class antibiotic, wherein the composition is in the sustained release form, timed release form, pulsatile release form, prolonged release form, extended release form or delayed release form, or a combination thereof. The composition can also additionally comprise an immediate release composition.

The compositions can be prepared in an easy and cost effective manner.

In another embodiment, a controlled release pharmaceutical composition of tetracycline-class antibiotic comprising tetracycline antibiotic, one or more bioadhesive polymer(s) and one or more pharmaceutically acceptable excipient(s) wherein said composition is formulated to increase the residence time of said pharmaceutical composition and/or tetracycline-class antibiotic in the gastrointestinal tract having an adhesive strength, measured as a force of detachment, of at least 100 mN when measured using advanced force gauge equipment (manufactured by Mecmesin, West Sussex, England).

In another embodiment, a controlled release pharmaceutical composition of tetracycline-class antibiotic, wherein composition releases from about 10% to about 35% of tetracycline-class antibiotic in one hour, not less than about 35% in four hours, from about 40% to about 85% of tetracycline-class antibiotic in six hours, not less than 70% in fourteen hours measured using USP Type II dissolution apparatus in 900 ml of 0.1 N HCl at 75 rpm.

In another embodiment, a controlled release pharmaceutical composition of minocycline, wherein composition releases from about 10% to about 35% of minocycline in one hour, from about 40% to about 85% of minocycline in six hours, measured using USP Type II dissolution apparatus in 900 ml of 0.1 N HCl at 75 rpm.

In another embodiment, specification discloses the method for reducing the incidence or severity of vestibular side effects resulting from the treatment of acne by the use of controlled release pharmaceutical composition of Minocycline, one or more bioadhesive polymer(s) and one or more pharmaceutically acceptable excipient(s) wherein said composition is formulated to increase the residence time of said pharmaceutical composition and/or minocycline in the gastrointestinal tract, such that:
i) said composition provides therapeutic blood concentration of minocycline over a 24 hours period
iii) said composition provides a peak blood plasma level (Cₘₐₓ) of minocycline in more than 4 hours (Tₘₐₓ).

In another embodiment, invention provides the method for reducing the incidence or severity of vestibular side effects resulting from the treatment of acne by the use of controlled release pharmaceutical composition of Minocycline, one or more bioadhesive polymer(s) and one or more pharmaceutically acceptable excipient(s) wherein said composition is formulated to increase the residence time of said pharmaceutical composition and/or minocycline in the gastrointestinal tract, such that:
ii) said composition provides therapeutic blood concentration of minocycline over a 24 hours period
iii) said composition provides a peak blood plasma level (Cₘₐₓ) of minocycline in about 4 hours to about 12 hours (Tₘₐₓ).

In another embodiment, the pharmacokinetic profile of a controlled release minocycline composition described herein, is pharmacokinetically distinct from marketed compositions of Minocycline like Minocin® (immediate release composition), Solodyn® (extended release composition). The pharmacokinetic distinctness may be due to, for example, a difference in the Cₘₐₓ, AUC (Area under curve)₍₀₋₇₂₎, and/or Tₘₐₓ parameters. The parameters may be single-dosage or steady-state. The Cₘₐₓ of a composition described herein may be less than about 80% and more preferably about 50% to about 80% of a marketed composition. The AUC₍₀₋₇₂₎ may be less than about 80% of a marketed composition. The Tₘₐₓ may be greater than by about 1.25 times to about 3.0 times of a marketed composition.

The foregoing examples are illustrative embodiments of the invention and are merely exemplary. A person skilled in the art may make variations and modifications without deviating from the spirit and scope of the invention. All such modifications and variations are intended to be included within the scope of the invention.

### EXAMPLES

### Example-1

| Ingredients | % w/w |
|---|---|
| Active | 20 |
| Sodium Alginate | 40 |
| Water | QS |
| Calcium chloride | 15 |
| Polyethylene oxide (PEO) | 10 |
| Sodium Carboxymethyl | 11 |
| Cellulose (NaCMC) | |
| Colloidal silicon dioxide | 2 |
| Magnesium stearate | 2 |

### Procedure

i) Sodium alginate is suspended in water and active was suspended in this colloidal solution.
ii) Calcium Chloride is dissolved in water and kept aside.
iii) Add step (i) into step (ii) dropwise to make beads under stirring, further filter the solution to separate the beads and dry the beads.
iv) Mix the dried beads with xanthan gum and sodium alginate.
v) Lubricate the beads of step (iv) with magnesium stearate and fill into capsules or sachets or filled in bottles with sweetening and flavouring agents as a powder for suspension.

### Example-2

| Ingredients | % w/w |
|---|---|
| Active | 25 |
| Diluents (e.g., Mannitol or | 40 |
| DCP or MCC) | |
| PEO | 20 |
| Polyvinyl Pyrolidone (PVP) | 10 |
| Isopropyl Alcohol (IPA) | QS |
| Magnesium Stearate | 2 |
| Colloidal silicon dioxide | 2 |

### Procedure:

i) Sift Active, Diluent, PVP and PEO through a suitable sieve.
ii) Granulate blend of step (i) with IPA.
iii) Dry the granules of step (ii) and sift through a suitable sieve.
iv) Lubricate the granules of step (iii) with magnesium stearate.
v) The bioadhesive granules of step (iv) can be further compressed into tablets using suitable diluents and lubricants or filled into capsules or sachets or filled into bottle with sweetening and flavouring agents as a powder for suspension.

### Example-3

| Ingredients | % w/w |
|---|---|
| Active | 20 |
| Diluent (MCC/DCP) | 40 |
| Sodium Alginate | 10 |
| Xanthan gum | 10 |
| NaCMC | 15 |
| Sodium bicarbonate | 4 |
| Sodium Lauryl Sulphate (SLS) | 1 |
| Water | QS |

### Procedure:

### Spheronization

i) Mix all the ingredients except sodium bicarbonate in a blender.
ii) Dissolve sodium bicarbonate in water.
iii) Granulate (ii) with (i)
iv) Wet mass of step (ii) is passed through Extruder and further spheronized to get the round pellets

The pellets can be filled into capsules, sachets or filled in bottles with sweetening and flavouring agents as a powder for suspension or compressed into tablets.

### Example 4

| Ingredients | % w/w |
|---|---|
| Active | 30 |
| Dliuents (e.g., Mannitol or | 30 |
| DCP or MCC) | |
| HPMC | 10 |
| Poloxamer | 10 |
| PEO | 10 |
| Colloidal silicon dioxide | 5 |
| Magnesium stearate | 5 |

### Procedure:

i) Sift Active, Diluent, HPMC, Poloxamer and PEO through a suitable sieve
ii) dry blend (i) in a blender.
iii) sift colloidal silicon dioxide and magnesium stearate through a suitable sieve.
iv) Lubricate (ii) with (iii) in a blender.
v) The blend is then compressed into tablets.

### Example 5

| Ingredients | % W/W |
|---|---|
| Active | 30 |
| Diluents (e.g. Mannitol or | 30 |
| DCP or MCC) | |
| HPMC | 10 |
| Poloxamer | 10 |
| Sodium Alginate | 10 |
| Colloidal silicon dioxide | 5 |
| Magnesium Stearate | 5 |

### Procedure:

i) Active, Diluents, HPMC, Poloxamer and sodium alginate are sifted through a suitable sieve.
ii) Step (i) is My blended in a blender.
iii) Lubricants are sifted through a suitable sieve and mixed with step (ii).
iv) Blend of step (iii) is then compressed into mini tablets.
v) These mini tablets can be filled into capsules

### Example 6

### B) First and Third Layer

| Ingredients | % W/W |
|---|---|
| Active | 5 |
| HPMC | 35 |
| PEO | 40 |
| Polycarbophil | 15 |
| Collodial Silicon Dioxide | 3 |
| Magnesium Stearate | 2 |

### Procedure:

i) Sift active, HPMC, PEO and Polycarbophil through a suitable sieve.
ii) dry blend (i) in a blender.
iii) sift colloidal silicon dioxide and magnesium stearate through a suitable sieve.
iv) Lubricate (ii) with (iii) in a blender.
v) Divide (iv) into equal portions for first and third layer.

### A) Middle Layer

| Ingredients | % W/W |
|---|---|
| Active | 70 |
| Diluents (E.g., Mannitol or | 16 |
| DCP or MCC) | |
| HPMC | 10 |
| Colloidal silicon dioxide | 2 |
| Magnesium Stearate | 2 |

### Procedure

i) Sift active, diluent and HPMC through a suitable sieve
ii) dry blend (i) in an blender/granulation can be done using IPA or water.
iii) sift colloidal silicon dioxide and magnesium stearate through a suitable sieve.
iv) Lubricate (ii) with (iii) in a blender.

Blends of A and B are compressed into trilayer tablets

### Example 7

| Ingredients | % W/W |
|---|---|
| Active | 20 |
| Diluents (e.g. Mannitol or | 25 |
| DCP or MCC) | |
| HPMC | 10 |
| PEO | 15 |
| Vitamin E TPGS* | 20 |
| Colloidal silicon dioxide | 3 |
| Magnesium Stearate | 2 |

*D-alpha-tocopheryl polyethylene glycol 1000 succinate

### Procedure

i) Mix active with Vitamin E TGPS at 70° C., cool to room temperature.
ii) Pulverize (i) using a suitable sieve
iii) Mix (ii) with all other excipients except the lubricants in a blender.
iv) Mix (iii) with magnesium stearate and colloidal silicon dioxide in a blender.
v) The blend is then compressed into tablets.

### Example 8

### B) First and Third Layer

| Ingredients | % W/W |
|---|---|
| Active | 5 |
| Hydroxypropylmethyl | 30 |
| cellulose (HPMC) | |
| PEO | 40 |
| Polyvinyl alcohol (PVA) | 10 |
| SLS | 10 |
| Collodial Silicon Dioxide | 3 |
| Magnesium Stearate | 2 |

### Procedure:

i) Sift active, HPMC, PEO, PVA and SLS through a suitable sieve.
ii) dry blend (i) in a blender.
iii) sift colloidal silicon dioxide and magnesium stearate through a suitable sieve.
iv) Lubricate (ii) with (iii) in a blender.
v) Divide (iv) into equal portions for first and third layer.
A) Middle layer

| Ingredients | % W/W |
|---|---|
| Active | 70 |
| Diluents (E.g., Mannitol or | 15 |
| DCP or MCC) | |
| HPMC | 10 |
| Colloidal silicon dioxide | 3 |
| Magnesium Stearate | 2 |

### Procedure

i) Sift active, diluent and HPMC through a suitable sieve
ii) dry blend (i) in a blender/granulation can be done using IPA or water.
iii) sift colloidal silicon dioxide and magnesium stearate through a suitable sieve.
iv) Lubricate (ii) with (iii) in a blender.

Blends of A and B are compressed into trilayer tablets

### Example 9

| Ingredients | % w/w |
|---|---|
| Active | 30 |
| Dliuents (e.g., Mannitol or | 30 |
| DCP or MCC) | |
| HPMC | 15 |
| PEO | 25 |
| Colloidal silicon dioxide | 3 |
| Magnesium stearate | 2 |
| Methylene chloride | QS |
| IPA | QS |

### Procedure:

i) Sift Active, Diluent, HPMC and PEO through a suitable sieve.
ii) dry blend (i) in a blender.
iii) Granulate (ii) with IPA:Methylene chloride.
iv) Dry (iii) in fluid bed dryer.
v) Granules obtained in (iv) are sifted through a suitable sieve.
vi) Sift Colloidal silicon dioxide and magnesium stearate through suitable sieve
vii) Lubricate (v) with (vi) in a blender.
v) The blend is then compressed into tablets

### Example 10

| Ingredients | % w/w |
|---|---|
| Active | 40 |
| Dliuents (e.g., Mannitol or | 10 |
| DCP or MCC) | |
| HPMC | 20 |
| PEO | 20 |
| NaCMC | 5 |
| Colloidal silicon dioxide | 3 |
| Magnesium stearate | 2 |

### Procedure

i) Sift Active, Diluent, HPMC, NaCMC and PEO through a suitable sieve
ii) dry blend (i) in a blender.
iii) Sift Colloidal silicon dioxide and magnesium stearate through a suitable sieve
iv) Add half quantity of (iii) to (ii) and mix in a blender.
v) Compact blend of (iv) using a roller compactor at a pressure.
vi) sift (v) through suitable sieve to obtain granules
vii) Mix remaining quantity of (iii) and (vi) in a blender.
viii) Blend of (vii) is compressed into tablets.

### Example 11

| Ingredients | % w/w |
|---|---|
| Active | 50 |
| Dliuents (e.g., Mannitol or | 15 |
| DCP or MCC) | |
| HPMC | 10 |
| PEO | 15 |
| Guar gum | 5 |
| Colloidal silicon dioxide | 3 |
| Magnesium stearate | 2 |
| Water | QS |

### Procedure:

i) Sift active, diluent, HPMC, guar gum and PEO through a suitable sieve
ii) dry blend (i) in a blender.
iii) Granulate (ii) with water and dry the wet mass in fluid bed dryer.
iv) Granules obtained in (iii) are sifted through a suitable sieve.
v) Sift Colloidal silicon dioxide and magnesium stearate through a suitable sieve.
vi) Lubricate (iv) with (v).
vii) Blend of step (vi) is then compressed into tablets.

### Example 12

### A) First Layer—

| Ingredients | % w/w |
|---|---|
| Active | 40 |
| Dliuents (e.g., Mannitol or | 25 |
| DCP or MCC) | |
| HPMC | 30 |
| Colloidal silicon dioxide | 3 |
| Magnesium stearate | 2 |
| IPA | QS |
| Methylene chloride | OS |

i) Sift active, diluent and HPMC through a suitable sieve and mix in a blender.
iii) Granulate (i) with IPA:methylene chloride, and dry the wet mass in a fluid bed dryer.
iii) Granules of (iii) passed through a suitable sieve.
iv) Sift colloidal silicon dioxide and magnesium stearate through a suitable sieve
v) Lubricate (iv) with (v).

### B) Second Layer—

| Ingredients | % w/w |
|---|---|
| HPMC | 30 |
| PEO | 35 |
| Dliuents (e.g., Mannitol or | 25 |
| DCP or MCC) | |
| Colloidal silicon dioxide | 5 |
| Magnesium stearate | 5 |

i) Sift HPMC, diluent and PEO through a suitable sieve.
ii) dry blend (i) in a blender.
iii) sift colloidal silicon dioxide and magnesium stearate through a suitable sieve.
iv) Lubricate (ii) with (iii) in a blender.

Compress both A and B to form bilayer tablet.

### Example 13

### B) First and Third Layer

| Ingredients | % W/W |
|---|---|
| HPMC | 40 |
| Xanthan gum | 20 |
| Polycarbophil | 25 |
| Colloidal silicon dioxide | 5 |
| Magnesium stearate | 5 |

### Procedure:

i) Sift HPMC, Xanthan gum and Polycarbophil through a suitable sieve.
ii) dry blend (i) in a blender.
iii) sift colloidal silicon dioxide and magnesium stearate through a suitable sieve.
iv) Lubricate (ii) with (iii) in a blender.
A) Middle layer

| Ingredients | % W/W |
|---|---|
| Active | 50 |
| Diluents (e.g., Mannitol or | 15 |
| DCP or MCC) | |
| HPMC | 10 |
| Colloidal silicon dioxide | 7 |
| Magnesium stearate | 3 |
| Water | QS |

i) Sift active, diluent, and HPMC through a suitable sieve
ii) dry blend (i) in a blender.
iii) Granulate (i) with (ii) and dry the wet mass in fluid bed dryer
iv) Granules obtained in (iii) are sifted through a suitable sieve.
v) Sift Colloidal silicon dioxide and magnesium stearate through a suitable sieve.
vi) Lubricate (iv) with (v).
vii) Blend of A and B is then compressed into trilayered tablet.

### Example 14

### B) First and Third Layer

| Ingredients | % W/W |
|---|---|
| HPMC | 40 |
| Xanthan gum | 20 |
| PEO | 30 |
| Colloidal silicon dioxide | 5 |
| Magnesium stearate | 5 |

### Procedure:

i) Sift HPMC, Xanthan gum and PEO through a suitable sieve
ii) dry blend (i) in a blender.
iii) sift colloidal silicon dioxide and magnesium stearate through a suitable sieve.
iv) Lubricate (ii) with (iii) in a blender.

### A) Middle Layer

| Ingredients | % W/W |
|---|---|
| Active | 40 |
| Diluents (e.g., Mannitol or | 10 |
| DCP or MCC) | |
| HPMC | 15 |
| Colloidal silicon dioxide | 3 |
| Magnesium stearate | 2 |
| IPA | QS |
| Methylene chloride | QS |

### Procedure:

i) Sift active, diluent, and HPMC through a suitable sieve.
ii) dry blend (i) in a blender.
iii) Granulate (ii) with IPA:methylene chloride and dry the wet mass in fluid bed dryer.
iv) Granules obtained in (iii) are sifted through a suitable sieve.
v) Sift Colloidal silicon dioxide and magnesium stearate through a suitable sieve.
vi) Lubricate (iv) with (v).
viii) Blend A and B are then compressed into trilayer tablet.

### Example 15

### A) First Layer

| Ingredients | % w/w |
|---|---|
| Active | 35 |
| HPMC | 15 |
| PEO | 20 |
| Dliuents (e.g., Mannitol or | 25 |
| DCP or MCC) | |
| Colloidal silicon dioxide | 2 |
| Magnesium stearate | 3 |
| Water | QS |

### Procedure

i) Sift active, diluent, HPMC and PEO through a suitable sieve and mix in a blender.
ii) granulate (i) with water and dry the wet mass in a fluid bed dryer.
iii) granules of (ii) passed through a suitable sieve.
iv) sift colloidal silicon dioxide and magnesium stearate through a suitable sieve
v) lubricate (iv) with (v).

### B) Second Layer

| Ingredients | % w/w |
|---|---|
| HPMC | 40 |
| Polyethylene Oxide (PEO) | 35 |
| Carbomer | 15 |
| Colloidal silicon dioxide | 5 |
| Magnesium stearate | 5 |

i) Sift HPMC, carbomer and PEO through a suitable sieve
ii) dry blend (i) in a blender.
iii) sift colloidal silicon dioxide and magnesium stearate through a suitable sieve.
iv) Lubricate (ii) with (iii) in a blender.

Blends of A and B are then compressed into bilayer tablets.

### Example 16

### A) First Layer

| Ingredients | % w/w |
|---|---|
| Active | 30 |
| HPMC | 30 |
| Xanthan gum | 10 |
| Dliuents (e.g., Mannitol or | 25 |
| DCP or MCC) | |
| Colloidal silicon dioxide | 3 |
| Magnesium stearate | 2 |

i) Sift active, diluent, HPMC and xanthan gum through a suitable sieve
ii) dry blend (i) in a blender.
iii) sift colloidal silicon dioxide and magnesium stearate through a suitable sieve.
iv) Lubricate (ii) with (iii) in a blender.

### B) Second Layer

| Ingredients | % w/w |
|---|---|
| HPMC | 35 |
| Polyethylene Oxide (PEO) | 35 |
| Dliuents (e.g., Mannitol or | 20 |
| DCP or MCC) | |
| Colloidal silicon dioxide | 5 |
| Magnesium stearate | 5 |

i) Sift HPMC, diluent and PEO through a suitable sieve
ii) dry blend (i) in a blender.
iii) sift colloidal silicon dioxide and magnesium stearate through a suitable sieve.
iv) Lubricate (ii) with (iii) in a blender.

Blends of A and B are then compressed into bilayer tablets.

### Example 17

### B) First and Third Layer

| Ingredients | % W/W |
|---|---|
| HPMC | 40 |
| Xanthan gum | 20 |
| Polycarbophil | 25 |
| Colloidal silicon dioxide | 5 |
| Magnesium stearate | 5 |

### Procedure:

i) Sift HPMC, Xanthan gum and Polycarbophil through a suitable sieve
ii) dry blend (i) in a blender.
iii) sift colloidal silicon dioxide and magnesium stearate through a suitable sieve.
iv) Lubricate (ii) with (iii) in a blender.

### A) Middle Layer

| Ingredients | % W/W |
|---|---|
| Active | 40 |
| Diluents (e.g., Mannitol or | 20 |
| DCP or MCC) | |
| HPMC | 15 |
| PEO | 15 |
| Colloidal silicon dioxide | 7 |
| Magnesium stearate | 3 |
| Water | QS |

i) Sift active, diluent, HPMC and PEO through a suitable sieve.
ii) dry blend (i) in a blender.
iii) Granulate (ii) with water and dry the wet mass in fluid bed dryer.
iv) Granules obtained in (iii) are sifted through a suitable sieve.
v) Sift Colloidal silicon dioxide and magnesium stearate through a suitable sieve.
vi) Lubricate (iv) with (v).
vii) Blend of A and B is then compressed into trilayered tablets.

### Example 18

### A) First Layer

| Ingredients | % w/w |
|---|---|
| Active | 70 |
| HPMC | 5 |
| Diluents (e.g., Mannitol or | 21 |
| DCP or MCC) | |
| Colloidal silicon dioxide | 2 |
| Magnesium stearate | 2 |

### Procedure:

i) Sift active, diluent, HPMC and diluent through a suitable sieve.
ii) dry blend (i) in a blender.
iii) sift colloidal silicon dioxide and magnesium stearate through a suitable sieve.
iv) Lubricate (ii) with (iii) in a blender.

### B) Second Layer

| Ingredients | % w/w |
|---|---|
| Active | 40 |
| HPMC | 35 |
| Diluents (e.g., Mannitol or | 20 |
| DCP or MCC) | |
| Colloidal silicon dioxide | 3 |
| Magnesium stearate | 2 |

i) Sift active, diluent, HPMC and diluent through a suitable sieve.
ii) dry blend (i) in a blender.
iii) sift colloidal silicon dioxide and magnesium stearate through a suitable sieve.
iv) Lubricate (ii) with (iii) in a blender.

### C) Third Layer

| Ingredients | % w/w |
|---|---|
| HPMC | 40 |
| Xanthan gum | 20 |
| PEO | 30 |
| Colloidal silicon dioxide | 5 |
| Magnesium stearate | 5 |

i) Sift xanthan gum, PEO and HPMC through a suitable sieve.
ii) dry blend (i) in a blender.
iii) sift colloidal silicon dioxide and magnesium stearate through a suitable sieve.
iv) Lubricate (ii) with (iii) in a blender.

Blend of A, B and C is then compressed into trilayered tablets.

### Example 19

### A) First Layer

| Ingredients | % w/w |
|---|---|
| Active | 50 |
| Hydroxypropylmethyl | 20 |
| cellulose) HPMC | |
| Carbomer | 10 |
| Dliuents (e.g., Mannitol or | 15 |
| DCP or MCC) | |
| Colloidal silicon dioxide | 2 |
| Magnesium stearate | 3 |

### Procedure:

i) Sift active, HPMC, carbomer and diluents through a suitable sieve
ii) dry blend (i) in a blender.
iii) sift colloidal silicon dioxide and magnesium stearate through a suitable sieve.
iv) Lubricate (ii) with (iii) in a blender.

### B) Second Layer

| Ingredients | % w/w |
|---|---|
| HPMC | 30 |
| Polyethylene Oxide (PEO) | 40 |
| Xanthan gum | 20 |
| Colloidal silicon dioxide | 2 |
| Magnesium stearate | 3 |

i) Sift HPMC, xanthan gum and PEO through a suitable sieve
ii) dry blend (i) in a blender.
iii) sift colloidal silicon dioxide and magnesium stearate through a suitable sieve.
iv) Lubricate (ii) with (iii) in a blender.

Blends of A and B are then compressed into bilayer tablets.

### Example 20

| Ingredients | % w/w |
|---|---|
| Active | 2 |
| Diluents (e.g. Mannitol or | 15 |
| DCP or MCC) | |
| HPMC | 30 |
| PEO | 45 |
| Stabilizing agent | 5 |
| Colloidal silicon dioxide | 2 |
| Magnesium Stearate | 1 |

### Procedure:

i) Active, Diluents, HPMC, stabilizing agent and PEO are sifted through a suitable sieve.
ii) Step (i) is dry blended in a blender.
iii) Lubricants are sifted through a suitable sieve and mixed with step (ii).
iv) Blend of step (iii) is then compressed into tablets.

### Example 21

| Ingredient | % w/w |
|---|---|
| Active IR Layer | |
| Minocycline HCl | 3 |
| Lactose Monohydrate | 10 |
| Microcrystalline cellulose | 5 |
| Croscarmellose Sodium | 1 |
| Magnesium Stearate | 0.2 |
| Active CR Layer | |
| Minocycline HCl | 8 |
| Hydroxypropylmethyl | 12 |
| cellulose) HPMC | |
| Lactose Monohydrate | 35 |
| Magnesium Stearate | 1 |
| Bioadhesive Layer | |
| Polyethylene Oxide | 10 |
| Hydroxypropylmethyl | 10 |
| cellulose) HPMC | |
| Lactose Monohydrate | 5 |
| Colloidal Silicon dioxide | 1 |
| Magnesium Stearate | 0.3 |

### Procedure:

### CR Drug Layer—

1) Sift minocycline hydrochloride, HPMC, polyethylene oxide, microcrystalline cellulose and lactose monohydrate through suitable sieve.
2) Lubricate the granules of step 1 with magnesium stearate.

### Bioadhesive Layer—

3) Sift and mix polyethylene oxide, HPMC, Lactose Monohydrate, Croscarmellose Sodium and Colloidal Silicon dioxide through suitable sieve.
4) Lubricate the above blend of step 3 with magnesium stearate.

### IR Layer—

5) Sift minocycline hydrochloride, lactose monohydrate, microcrystalline cellulose and lactose through suitable sieve.
6) Lubricate the granules of step 5 with magnesium stearate.
7) Compress the lubricated blend of step 2 and step 4 followed by step 6 on top of CR layer into trilayer tablets using suitable shaped punches and dies (13.3 mm round punch).

### Example 22

| Ingredient | % w/w |
|---|---|
| Active CR Layer | |
| Minocycline HCl | 10.4 |
| Hydroxypropylmethyl | 10.4 |
| cellulose | |
| Lactose Monohydrate | 20.7 |
| Lactose Monohydrate | 31.1 |
| Magnesium Stearate | 0.5 |
| Bioadhesive Layer | |
| Polyethylene Oxide | 10.4 |
| Hydroxypropylmethyl | 10.4 |
| cellulose | |
| Lactose Monohydrate | 5.4 |
| Colloidal Silicon dioxide | 0.5 |
| Magnesium Stearate | 0.3 |

### Procedure:

### CR Drug Layer—

1) Sift minocycline hydrochloride, HPMC, polyethylene oxide, microcrystalline cellulose and lactose monohydrate through suitable sieve.
2) Lubricate the granules of step 1 with magnesium stearate.

### Bioadhesive Layer—

3) Sift and mix polyethylene oxide, HPMC, Lactose Monohydrate, Croscarmellose Sodium and Colloidal Silicon dioxide through suitable seive.
4) Lubricate the above blend of step 3 with magnesium stearate.
5) Compress the lubricated blend of step 2 and step 4 into bilayer tablets using suitable shaped punches and dies (13.3 mm round punch).

### Determination of Bioadhesion:

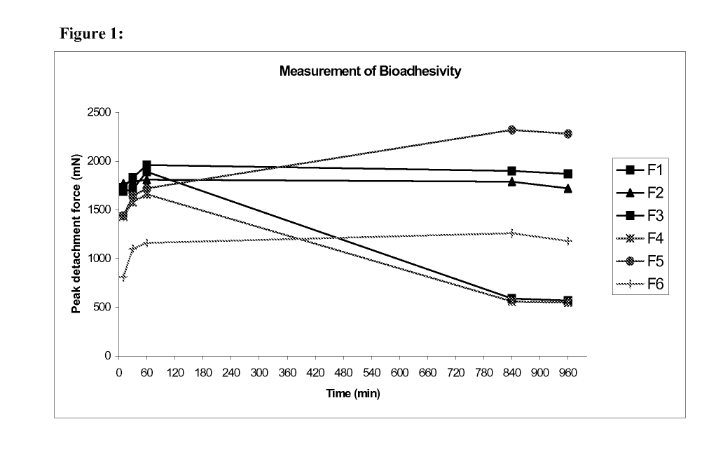

Bioadhesion was determined by tensiometric method. For the determination, an advanced force gauge equipment (mfg. by Mecmesin, West Sussex, England) was used. Freshly excised Sheep intestinal tissue was taken and stored in a Tyrode solution at 4.degree. C. until used for the experiment. The tissue was cut into pieces (3.times.4 cm) and mounted on the glass slide and tightened with a thread. 0.5 ml Phosphate buffered saline (PBS) was placed on the tissue. The bioadhesive tablet prepared as in examples 1 to 6, was placed on this tissue and another 0.5 ml PBS was placed on the tablet. A glass slide with a 10 g weight was placed on the tablet and it was allowed to hydrate for 10 min., 30 min., 60 min., 840 and 960 min. At the specific time interval, the hydrated tablet along with slide was mounted on the stage of the bioadhesion apparatus. Probe was then lowered at fixed speed of 0.2 mm/sec. and upper slide was attached to the hook of the probe by means of a thread. The peak detachment force was considered as the bioadhesive force. The force required to separate the tablet from biological substrate was recorded in mN as demonstrated in accompanying FIG. 1.

### Formula 1:

| Sr. No. | Ingredients | % w/w |
|---|---|---|
| 1. | PEO | 48.86 |
| 2. | MCC | 48.86 |
| 3. | Aerosil 200 | 1.36 |
| 4. | Mg-stearate | 0.91 |

### Formula 2:

| Sr. No. | Ingredients | % w/w |
|---|---|---|
| 1. | HPMC K 100M | 48.86 |
| 2. | MCC | 48.86 |
| 4. | Aerosil 200 | 1.36 |
| 5. | Mg-stearate | 0.91 |

### Formula 3:

| Sr. No. | Ingredients | % w/w |
|---|---|---|
| 1. | PEO | 48.86 |
| 2. | Xanthan gum | 48.86 |
| 4. | Aerosil 200 | 1.36 |
| 5. | Mg-stearate | 0.91 |

### Formula 4:

| Sr. No. | Ingredients | % w/w |
|---|---|---|
| 1. | Xanthan gum | 48.86 |
| 2. | MCC | 48.86 |
| 3. | Aerosil 200 | 1.36 |
| 4. | Mg-stearate | 0.91 |

### Formula 5:

| Sr. No. | Ingredients | % w/w |
|---|---|---|
| 1. | PEO | 48.86 |
| 2. | Sodium CMC | 48.86 |
| 4. | Aerosil 200 | 1.36 |
| 5. | Mg-stearate | 0.91 |

Formula 6:

| Sr. No. | Ingredients | % w/w |
|---|---|---|
| 1. | Sodium CMC | 48.86 |
| 2. | MCC | 48.86 |
| 4. | Aerosil 200 | 1.36 |
| 5. | Mg-stearate | 0.91 |

### In-Vitro Dissolution Study:

Table 1 given below shows the dissolution profile of Minocycline Hydrochloride

Controlled Release Tablets of Example 21 of the present invention carried out in 900 ml of 0.1N HCl for 24 hours using Apparatus USP-II (Paddle) at 75 rpm speed. The release profile (cumulative % of drug released) is given in Table 1.

TABLE 1

| | Cumulative % Drug |
|---|---|
| Time | Release for |
| in Hrs | Example 21 |
| 1 | 10-35% |
| 4 | Not Less Than 35% |
| 6 | 40-85% |
| 14 | Not Less Than 70% |
| 18 | Not Less Than 80% |

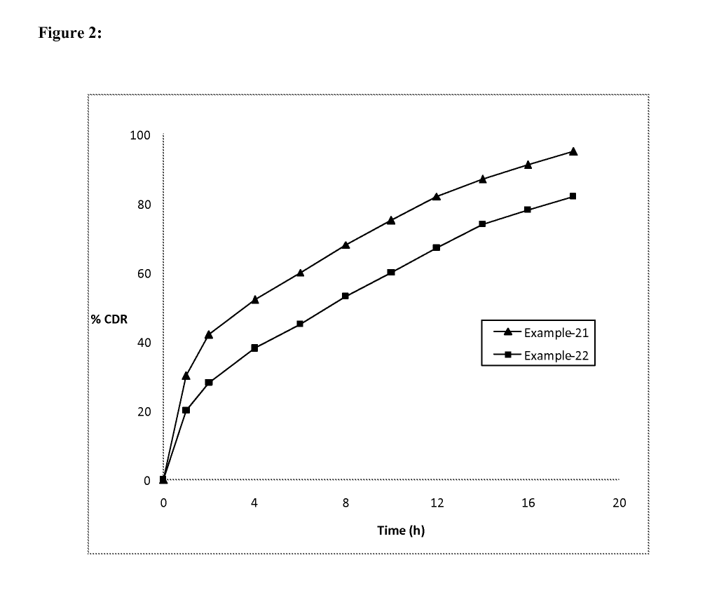

FIG. 2 discloses dissolution profile of Example 21 & 22, a graph of cumulative percent drug release vs time.

1. A controlled release pharmaceutical composition comprising tetracycline-class antibiotic, one or more bioadhesive polymer(s) and one or more pharmaceutically acceptable excipient(s) wherein said composition is formulated to increase the residence time of said pharmaceutical composition and/or tetracycline-class antibiotic in the gastrointestinal tract.

2. The controlled release pharmaceutical composition of claim 1, wherein tetracycline-class antibiotic is minocycline or a pharmaceutically acceptable salt thereof.

3. The controlled release pharmaceutical composition of claim 1, wherein bioadhesive polymer(s) is selected from polycarbophils, carbomers, lectins, pectine, zein, modified zein, casein, gelatin, gluten, serum albumin, collagen, chitosan, oligosaccharides and polysaccharides such as cellulose their derivatives such as methyl cellulose, ethyl cellulose, hydroxypropyl cellulose, hydroxypropylmethyl cellulose, hydroxybutylmethyl cellulose, cellulose acetate, cellulose propionate, cellulose acetate butyrate, cellulose acetate phthalate, carboxymethyl cellulose, cellulose triacetate, cellulose sulfate sodium salt, dextrans, tamarind seed polysaccharide, gellan, carrageenan; hyaluronic acid, polyhyaluronic acid, alginic acid, sodium alginate; gums like xanthan gum, guar gum, gum Arabic locust bean gum; polyvinylacetate, polyvinylalcohol, povidone/polyethylene oxide, acrylic and methacrylic acid their copolymers, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), polymers having a hydrophobic backbone with at least one hydrophilic group pendant from the backbone, polymers having a hydrophobic backbone with at least one hydrophobic group pendant from the backbone, and blends and copolymers or mixtures thereof.

4. The controlled release pharmaceutical composition of claim 1, wherein increase in residence time in gastrointestinal tract is achieved by bioadhesion, and/or by delaying expulsion from gastrointestinal tract.

5. The controlled release pharmaceutical composition of claim 1 is tablet selected from single layered tablets, multilayered tablets, mini tablets, bioadhesive tablets, caplets, matrix tablets, tablet within a tablet and mucoadhesive tablets.

6. A controlled release pharmaceutical composition of tetracycline-class antibiotic comprising: at least two entities selected from
a. controlled release entity
b. bioadhesive entity
c. optionally one or more immediate release entities; and one or more pharmaceutically acceptable excipient(s), wherein said composition is formulated to increase the residence time of said pharmaceutical composition and/or tetracycline-class antibiotic in the gastrointestinal tract.

7. The controlled release pharmaceutical composition of claim 9 is multi-layered tablet.

8. A controlled release pharmaceutical composition of Minocycline, wherein composition releases from about 10% to about 35% of Minocycline in one hour, from about 40% to about 85% of Minocycline in six hours, measured using USP Type II dissolution apparatus in 900 ml of 0.1 N HCl at 75 rpm.

9. A method for reducing the incidence or severity of vestibular side effects resulting from the treatment of acne by the use of controlled release pharmaceutical composition of claim 2, wherein said composition provides therapeutic blood concentration of minocycline over a 24 hours period and a peak blood plasma level (Cₘₐₓ) of minocycline in more than 4 hours (Tₘₐₓ).

10. The method for reducing the incidence or severity of vestibular side effects resulting from the treatment of acne by the use of controlled release pharmaceutical composition of claim 2, wherein said composition provides therapeutic blood concentration of minocycline over a 24 hours period and a peak blood plasma level (Cₘₐₓ) of minocycline in about 4 to about 12 hours (Tₘₐₓ).
